(19) United States
(12) Patent Application Publication
(10) Pub. No.: US 2026/0216230 A1
(43) Pub. Date: Jul. 30, 2026

(54) SERRATIA ANTICANCER POLYSACCHARIDE COMPOSITIONS AND METHODS

(71) Applicant: Board of Supervisors of Louisiana State University and Agricultural and Mechanical College, Baton Rouge, LA (US)

(72) Inventor: Roger A. LAINE, Baton Rouge, LA (US)

(21) Appl. No.: 19/146,615

(22) PCT Filed: Jan. 11, 2024
(86) PCT No.: PCT/US2024/011169

§ 371 Date: Jul. 9, 2025

Related U.S. Application Data
(60) Provisional application No. 63/479,559, filed on Jan. 12, 2023.

(51) Int. Cl. A61K 31/715; A61K 45/06; A61P 35/00; C08B 37/00; C12P 19/04

(52) U.S. Cl. CPC A61K 31/715; A61K 45/06; A61P 35/00; C08B 37/0003; C12P 19/04

(57) ABSTRACT

In one aspect, the disclosure relates to compounds derived from Serratia anticancer polysaccharide (PS1), methods of making the same, pharmaceutical compositions comprising the same, and methods of treating tumors using same. In one aspect, the disclosed compounds contain a repeating polysaccharide unit and a lipid linked to the polysaccharide either by an ester bond or a phosphodiester bond. In another aspect, the disclosed compounds and methods are effective against a variety of tumor types through the mechanism of tumor capillary hemorrhage and do not cause systemic side effects such as, for example, antibody-mediated immune responses in subjects. This abstract is intended as a scanning tool for purposes of searching in the particular art and is not intended to be limiting of the present disclosure.

### CROSS REFERENCE TO RELATED APPLICATIONS

This application claims the benefit of U.S. Provisional Application No. 63/479,559 filed on Jan. 12, 2023, which is incorporated herein by reference in its entirety.

### BACKGROUND

Additional approaches to cancer therapy are a global need. Unique Streptococcus and Serratia bacterial extracts are known to have tumor-specific capillary destructive activity, knowledge of which originates with observations that certain sarcomas were cured by erysipelas infections as early as the 1860s. “Coley's Toxin” was a mixture of a heat treated cell-free culture medium of Streptococcus pyogenes and Serratia marcescens used successfully for tumor treatment from 1890 to 1920, until the advent of radiation therapy. Coley's toxin was produced by Parke-Davis and Co. for oncology use, though Parke-Davis's batches were inconsistent in potency due to the lack of information on the active pharmaceutical ingredients (API). The API from Streptococcus was not investigated, but the API from Serratia was pursued until 1943 by Shear and co-workers. Thus, the last chemical work on the active principle from the Serratia marcescens component of “Coley's Toxin” was performed in 1943, using hemorrhage in mouse tumors as an assay. The component was a peptide-free polysaccharide and putatively had a fatty acid and phosphate covalently attached suggesting a phospholipid attachment. No molecular weight or structure analysis was performed due to lack of technology, although a sugar composition analysis was performed by primitive colorimetric methods available at the time. In 1947 it was demonstrated in a mouse in vivo tumor by Algire that specific tumor capillary destruction was the mechanism of action.

In an independent advance in this area in the 1980's, Hellerqvist, et al. isolated a Streptococcus agalactica polysaccharide “CM101” as the etiologic agent of pulmonary disease in sheep, and of “Early Onset Disease” in human neonates, causing neutrophil attack specifically on rapidly growing neonate lung capillaries for 5 days after birth. Correctly guessing that a different type of capillary was hypoxically stimulated in neonate's lungs, Hellerqvist tested CM101 on mouse tumors, because tumors are hypoxic, and found CM101 specifically targeted tumor capillaries in 4 mouse models, with no side effects. This Group B streptococcus (GBS) polysaccharide fraction “CM101” was approved by the FDA as the test item for IND4578. In the Phase I trial, CM101 had a remarkable 33% effectivity in a safety trial of 15 mixed, refractory stage 4 cancer patients, with one cure. This level of effectivity would pass a Phase II trial today. (Taxol had 12% effectivity in a phase I, with refractory Stage 4 patients). CM101 development was a victim of business and finance errors, and was not followed into a planned Phase II.

Significantly, after the phase I clinical trial, Hellerqvist's group succeeded isolating and cloning a CM101 binding receptor, 55 kDa protein (SP55) from sheep capillary endothelium. Using PCR, they discovered a 59 kDa human homolog (HP59) in humans encoded by the SLC17A5 gene. HP59 was, by monoclonal antibody, expressed on 25 tested human tumors in capillary endothelium. Meanwhile, Serratia anticancer polysaccharide (PS1) is a tumor vascular disruptor, specific for tumor capillaries. The APIs of GBS toxin CM101 and PS1 Serratia polysaccharide have not been characterized as to their specific pharmacophores. Although it is believed that CM101 and PS1 may have some structural similarities and/or similar properties, interaction of these CM101 binding receptors with PS1 remains uninvestigated, and specific PS1 receptors have not yet been identified. Since CM101 does not elicit an antibody-based immune response in humans even after as many as 15 infusions, and since CM101 side effects were not observed in Phase I clinical trials, it is possible that one or more components or fractions of PS1 may form the basis of a safe and effective cancer therapeutic, but this clinical space remains unexplored.

Therapeutics that currently address angiogenesis include Avastin and Erbutux, among numerous other examples. These technologies use humanized monoclonal antibodies against vascular endothelial growth factor (VEGF) and its receptor (VEGFR), while other drugs in the arena address downstream signaling enzymes such as tyrosine kinase inhibitors. The markets for the anti-tumor angiogenesis group of drugs is estimated at 10 billion or more US dollars, typically costing about 100,000 US dollars per treatment. Tumors rapidly develop alternative signals to attract vasculature; thus these treatments are only used with other toxic chemotherapeutic reagents such as cis-platinum, and all can have serious side effects, including, but not limited to, perforated intestine. Such therapies added to the chemotherapeutic regimens only typically add 4-6 months of lifespan.

Despite advances in anticancer polysaccharide research, there is still a scarcity of compounds and compositions that are consistent in potency and effective in the treatment of various cancers, including late stage cancers, which are inexpensive to produce, and that are not associated with systemic side effects including, but not limited to, antibody-based immune response. Ideally, tumors treated with these compounds would be unable to develop alternative vasculature attraction strategies in patients undergoing treatment with these compounds. These needs and other needs are satisfied by the present disclosure.

### SUMMARY

In accordance with the purpose(s) of the present disclosure, as embodied and broadly described herein, the disclosure, in one aspect, relates to compounds derived from Serratia anticancer polysaccharide (PS1), methods of making the same, pharmaceutical compositions comprising the same, and methods of treating tumors using same. In one aspect, the disclosed compounds contain a repeating polysaccharide unit and a lipid linked to the polysaccharide either by an ester bond or a phosphodiester bond. In another aspect, the disclosed compounds and methods are effective against a variety of tumor types through the mechanism of tumor capillary hemorrhage and do not cause systemic side effects such as, for example, antibody-mediated immune responses in subjects.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows Sephacryl 400 size exclusion column (SEC) 30 cm×1 cm, 500 kDa dextran standard (first peak) centered on fraction 25 and PS1 polysaccharide (second peak), 250 kDa, centered on fraction 45. Note the broad peaks based on a Poisson distribution of polymer length. Polysaccharides have a broad MW distribution depending on the length of polymer synthesized. A broad peak may not indicate heterogeneity, but simply range of polymer length, a Poisson distribution. Note the broad peak from 500 kDa standard dextran. The column can be run in distilled water, 20% ethanol or 4% acetic acid, and the fractions containing PS1 recovered by lyophilization or other drying.

FIGS. 2A-2B show gel permeation chromatography (GPC) SEC for Serratia PS1 molecular weight determination: Size Exclusion Chromatography: A Waters UltraHydrogel 1000, 300×7.8 mm column (Waters Corp., Milford, MA) was used for the separation. The GPC used an Agilent 1100 pump (Agilent Technologies, Palo Alto, CA). For detection, a Wyatt Heleos Multi Angle Light Scattering (MALS) unit was used. Data acquisition and all the calculations were performed using Wyatt's Astra 6 software (Wyatt). For conventional SEC (calibration with dextran standards), a series of water solutions of 4 monodisperse dextrans. The refractive index increment dn/dC used for all the calculations was 0.148 mL/g. FIG. 2A is a refractive index tracing and FIG. 2B is light scattering, showing a small peak before the main peak as an aggregate, but the remainder of the broad PS1 peak appears monomolecular, with a Poisson distribution.

FIG. 3 shows a shows a sugar composition analysis of PS1 by methanolysis and acetylation of the methyl glycosides followed by gas-liquid chromatography. Mannose is the main component (8 units), with small amounts of rhamnose (1 unit), galactose (one unit), glucose (2 units), and galactosamine (1 unit) represented by their alditol acetates. A potential repeat structure of the PS1 glucan moiety based on methylation: α1,4Manₚ-α1,2Manₚ-α1,4Manₚ-α1,6(α1,2Manₚ)Manₚ (with the reducing end Manₚ branched on the 2 and 6 positions in this model). The terminal Man and 2,6-linked Man data represent branch points. The branch points could be on the 6-position of a 2-linked Man. Alternative repeat units could be as follows: α1,4Manₚ-α1,2Manₚ-α1,4(α1,6Manₚ)Manₚ-α1,2-α1,6Manₚ.

FIGS. 4A-4C show the left flank of Balb/c mice were injected subcutaneously with CT26 cells (2⁶/mice/100 μL). Tumor volumes were measured twice per week. Once tumors reached 300-500 mm³, mice were divided into the following 3 groups: A: Vehicle (N=10); B: CM101 (N=10); C: PS1 (N=10). Mice in each group were injected (i.v. tail vein) with the following A (FIG. 4A): Vehicle (N=10)—normal saline (5 μL/g), B (FIG. 4B) CM101 (N=10) 50 μg/kg, 5 L/g (1.2 μg/mouse), C (FIG. 4C) PS1 (N=10)—200 μg/kg, 5 L/g (4.8 μg/mouse). After 4 hr, all the mice were euthanized and tumor surgically removed and scored for vasculature leakage.

FIG. 5 shows polyacrylamide gel electrophoresis (PAGE) of Sialin expression. Clones 6 and 10 show as dimers in the gel with glycoform bands. From HeLa U937 cells, HA tag, anti-actin standard, Western Blot. WT6 expresses SP555, U937 clones 6k and 10 express HP59, SP55 and HP59 run in gels as dimers. Actin is not glycosylated and shows as a single band.

FIGS. 6A-6C show mass spectral analysis of PS1, where horizontal axis is m/z and vertical axis represents abundance (in arbitrary units). Peaks at 161.1 and 190.1 m/z in FIG. 6A represent 2-hexose or rhamnose. Major peaks in FIG. 6B represent a 2,6-hexose.

FIG. 7 shows an intersugar linkage analysis showing a 4-linked galactosamine, 2,6-linked mannose, 2-linked mannose, and 4-linked hexose. Horizontal lines represent fragmentation points in the molecule and numbers represent fragments appearing in mass spectra.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

In one aspect, disclosed herein is a method for producing an anticancer polysaccharide, the method including at least the steps of:
(a) extracting Serratia polysaccharide PS1 from a culture at least one strain of Serratia marcescens;
(b) subjecting the PS1 to at least one chemical or enzymatic treatment to hydrolyze at least one moiety from the PS1.

In another aspect, the method further optionally includes chromatographically separating at least one fraction of the PS1 from the culture prior to performing step (b) by a method such as, for example, size exclusion chromatography. In still another aspect, the at least one chemical treatment can be hydrazinolysis-deamination, Smith degradation using periodic acid, partial acid hydrolysis, acetolysis, or any combination thereof. In some aspects, the anticancer polysaccharide can be extracted using a solvent such as, for example, chloroform, dichloromethane, or a combination thereof.

Also disclosed herein are compounds produced by the disclosed methods. In one aspect, the compounds can include a plurality of polysaccharide units selected from α1,6Manₚ-α1,4Manₚ-α1,4(α1,2Manₚ)Manₚ, α1,4Manₚ-α1,2-Manₚ-α1,4(α1,6Manₚ)Manₚ-α1,2-α1,6Manₚ, or any combination thereof. In another aspect, the compounds can have a molar mass of about 250 kDa. In yet another aspect, the compound further includes at least one fatty acid covalently attached to at least one of the plurality of the polysaccharide units either via an ester linkage or a phosphodiester linkage.

In one aspect, the at least one fatty acid can be selected from decanoic acid, undecanoic acid, dodecanoic acid, tridecanoic acid, tetradecanoic acid, pentadecanoic acid, hexadecanoic acid, heptadecanoic acid, octadecanoic acid, hexadecenoic acid, octadecenoic acid, 3,OH-decanoic acid, 3,OH-5-dodecenoic acid, 3,OH-dodecanoic acid, methylenehexadecanoic acid, methyleneoctadecanoic acid, or any combination thereof.

In one aspect, disclosed herein are pharmaceutical compositions including the disclosed compounds, as well as methods for treating a tumor in a subject, the methods including at least the step of administering a disclosed compound or pharmaceutical composition to the subject. In a further aspect, from about 2 mg to about 10 mg of the compound can be administered to the subject per kilogram of body weight of the subject, or from about 2 mg to about 6 mg, or about 2.4 mg, or about 4.8 mg per kg of subject body weight. In another aspect, in the disclosed method, the compounds derived from PS1 cause capillaries supplying the tumor to hemorrhage. In another aspect, the methods do not cause systemic side effects in the subject.

In still another aspect, the tumor can be a bone tumor, a muscle sarcoma, a brain tumor, a breast tumor, a liver tumor, a gall bladder tumor, a colorectal tumor, a stomach tumor, a pancreatic tumor, an esophageal tumor, a bladder tumor, a prostate tumor, a uterine tumor, an ovarian tumor, an oral tumor, a salivary gland tumor, a kidney tumor, a skin tumor, a lung tumor, a prostate tumor, or any combination thereof. In an aspect, the tumor can be a colorectal tumor and at least 90% of the capillaries supplying the tumor hemorrhage. In one aspect, the method further includes administering at least one additional treatment to the subject such as, for example, surgery, radiation, administering at least one additional chemotherapeutic agent to the subject, or any combination thereof. In a further aspect, the additional treatment can be performed simultaneously with administration of the compound or composition, or before or after administration of the compound or composition.

In any of these aspects, the subject can be a mammal such as, for example, a human, cat, dog, horse, cattle, swine, goat, sheep, mouse, rat, hamster, guinea pig, or rabbit. In another aspect, the compound or pharmaceutical composition can be administered intravenously or intratumorally. In still another aspect, the method can be performed once or can be performed at least twice.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a receptor,” “a clone,” or “a monosaccharide,” include, but are not limited to, mixtures or combinations of two or more such receptors, clones, or monosaccharides, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y.’ The range can also be expressed as an upper limit, e.g. ‘about x, y, z, or less’ and should be interpreted to include the specific ranges of ‘about x,’ ‘about y’, and ‘about z’ as well as the ranges of ‘less than x’, less than y′, and ‘less than z’. Likewise, the phrase ‘about x, y, z, or greater’ should be interpreted to include the specific ranges of ‘about x,’ ‘about y,’ and ‘about z’ as well as the ranges of ‘greater than x,’ greater than y,′ and ‘greater than z.’ In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of an API refers to an amount that is sufficient to achieve the desired improvement in the property modulated by the formulation component, e.g. achieving the desired level of control or treatment for a disease or condition such as, for example, cancer. The specific level in terms of wt % in a composition required as an effective amount will depend upon a variety of factors including the type and stage of the cancer; whether the cancer has metastasized; concurrent treatment with any other anticancer therapeutics, surgeries, or radiation; age, sex, and weight of the subject, and the like.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### Aspects

The present disclosure can be described in accordance with the following numbered aspects, which should not be confused with the claims.

Aspect 1. A method for producing an anticancer polysaccharide, the method comprising:
(a) extracting Serratia polysaccharide PS1 from a culture at least one strain of Serratia marcescens;
(b) subjecting the PS1 to at least one chemical or enzymatic treatment to hydrolyze at least one moiety from the PS1.

Aspect 2. The method of aspect 1, further comprising chromatographically separating at least one fraction of the PS1 from the culture prior to performing step (b).

Aspect 3. The method of aspect 2, wherein chromatographically separating comprises size exclusion chromatography.

Aspect 4. The method of any one of aspects 1-3, wherein the at least one chemical treatment comprises hydrazinolysis-deamination, Smith degradation using periodic acid, partial acid hydrolysis, acetolysis, or any combination thereof.

Aspect 5. The method any one of aspects 1-4, further comprising extracting the anticancer polysaccharide using a solvent.

Aspect 6. The method of aspect 5, wherein the solvent comprises chloroform, dichloromethane, or any combination thereof.

Aspect 7. A compound produced by the method of any one of aspects 1-6.

Aspect 8. The compound of aspect 7, wherein the compound comprises a plurality of polysaccharide units selected from α1,6Manₚ-α1,4Manₚ-α1,4(α1,2Manₚ)Manₚ, α1,4Manₚ-α1,2-Manₚ-α1,4(α1,6Manₚ)Manₚ-α1,2-α1,6Manₚ, or any combination thereof.

Aspect 9. The compound of aspect 7 or 8, wherein the compound has a molar mass of about 250 kDa.

Aspect 10. The compound of any one of aspects 7-9, wherein the compound further comprises at least one fatty acid covalently attached to at least one of the plurality of the polysaccharide units.

Aspect 11. The compound of aspect 10, wherein the at least one fatty acid is covalently attached via an ester linkage or a phosphodiester linkage.

Aspect 12. The compound of aspect 10 or 11, wherein the at least one fatty acid comprises decanoic acid, undecanoic acid, dodecanoic acid, tridecanoic acid, tetradecanoic acid, pentadecanoic acid, hexadecanoic acid, heptadecanoic acid, octadecanoic acid, hexadecenoic acid, octadecenoic acid, 3,OH-decanoic acid, 3,OH-5-dodecenoic acid, 3,OH-dodecanoic acid, methylenehexadecanoic acid, methyleneoctadecanoic acid, or any combination thereof.

Aspect 13. A pharmaceutical composition comprising the compound of any one of aspects 7-12.

Aspect 14. A method for treating a tumor in a subject, the method comprising administering to the subject the compound of any one of aspects 7-12 or the pharmaceutical composition of aspect 13.

Aspect 15. The method of aspect 14, wherein from about 2 mg to about 10 mg of the compound are administered to the subject per kg of body weight of the subject.

Aspect 16. The method of aspect 15, wherein the compound causes capillaries supplying the tumor to hemorrhage.

Aspect 17. The method of aspect 15 or 16, wherein the method does not cause systemic side effects in the subject.

Aspect 18. The method of any one of aspects 14-17, wherein the tumor comprises a bone tumor, a muscle sarcoma, a brain tumor, a breast tumor, a liver tumor, a gall bladder tumor, a colorectal tumor, a stomach tumor, a pancreatic tumor, an esophageal tumor, a bladder tumor, a prostate tumor, a uterine tumor, an ovarian tumor, an oral tumor, a salivary gland tumor, a kidney tumor, a skin tumor, a lung tumor, a prostate tumor, or any combination thereof.

Aspect 19. The method of aspect 16, wherein the tumor is a colorectal tumor and wherein at least 90% of the capillaries supplying the tumor hemorrhage.

Aspect 20. The method of any one of aspects 14-19, further comprising administering at least one additional treatment to the subject.

Aspect 21. The method of aspect 20, wherein the at least one additional treatment comprises surgery, radiation, administering at least one additional chemotherapeutic agent to the subject, or any combination thereof.

Aspect 22. The method of aspect 20 or 21, wherein the at least one additional treatment is performed simultaneously with administration of the compound or composition.

Aspect 23. The method of aspect 20 or 21, wherein the at least one additional treatment is performed before or after administration of the compound or composition.

Aspect 24. The method of any one of aspects 14-23, wherein the subject is a mammal.

Aspect 25. The method of aspect 24, wherein the mammal is a human, cat, dog, horse, cattle, swine, goat, sheep, mouse, rat, hamster, guinea pig, or rabbit.

Aspect 26. The method of any one of aspects 14-25, wherein the compound or pharmaceutical composition is administered intravenously or intratumorally.

Aspect 27. The method of any one of aspects 14-26, wherein the method is performed once.

Aspect 28. The method of any one of aspects 14-26, wherein the method is performed at least twice.

### Examples

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Overview

Two different purification schemes from the 1940's have been repeated; in both cases a polysaccharide fraction was isolated that gives a broad peak centered at 250,000 Da on SEC and HPLC. Preliminary methylation linkage analysis shows terminal, 2-linked, 4-linked and 2,6-branched hexose and 4-linked glucosamine in the structure. With modern technology, it is proposed to characterize the structure of the polysaccharide fraction, seek the minimum pharmacophore, and determine if more active fractions exist within the broad GPC peak. In collaboration with Murigenics, Inc., Vallejo, CA, a mouse tumor hemorrhage C26 colon tumor model has been established in Balb/c mice. This mouse model demonstrated that the PS1 polysaccharide has the same tumor hemorrhage effect found for Group B Streptococcus agalactica (GBS) Toxin CM101 by Hellerqvist, which were directly compared. The interesting parallel is that the GBS active fraction has a molecular weight of 270,000 broad peak, and has a lipid and a phosphate attached, but a different sugar composition, however, with no structural work having been done to date. The GBS toxin caused hemorrhage in mouse tumors, and respiratory distress in a sheep pulmonary blood pressure assay. A Phase I human trial of CM101 had 33% effectivity, remarkable for stage 4 cancers. The receptor for the GBS toxin CM101 may be the same for the PS1 polysaccharide since only tumor vasculature was affected by both toxins.

Serratia polysaccharide PS1 was purified at scale for characterization and testing, including isotopic labeling through use of ¹³C glucose for ¹³C NMR analysis. Batches, subfractions, and fragments of PS1 were tested in a C26 mouse tumor hemorrhage assay to find the smallest active principle, which was confirmed using a cell-based PS1 binding and activity assay was conducted with SP55- and HP59-expressing HeLa cell lines. Sugar composition was determined by GC, GC-MS, and Dionex sugar analyses. Methylation linkage analysis was assessed using GC-MS in the main peak from collected SEC fractions to determine heterogeneity within the peak. Partial degradation was used to obtain oligosaccharides useful for pharmacophore structure including, but not limited to:
(i) hydrazinolysis-deamination reactions, partial acetolysis reactions, partial hydrolysis, and periodate use
(ii) purification of oligosaccharides generated in (i) by SEC, carbon-celite chromatography, TLC, and/or HPLC assays
(iii) sugar composition analysis using GC/MS of methyl glycosides, Dionex sugar analysis, and use of acetylated alditols
(iv) methylation linkage analysis allowed determination of non-reducing end sugar for partial glycosylhydrolase and enzyme degradation experiments
(v) sequential partial enzyme degradation was used to establish anomeric configuration, branch length, and sugar monomers required for activity
(vi) ¹³C enriched PS1 was used for NMR studies of oligosaccharides
(vii) site of attachment of putative phospholipid components was determined
(viii) minimum pharmacophore structure was determined

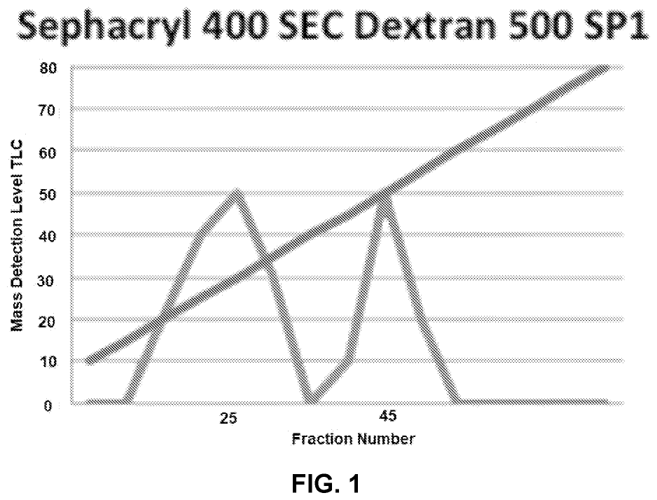

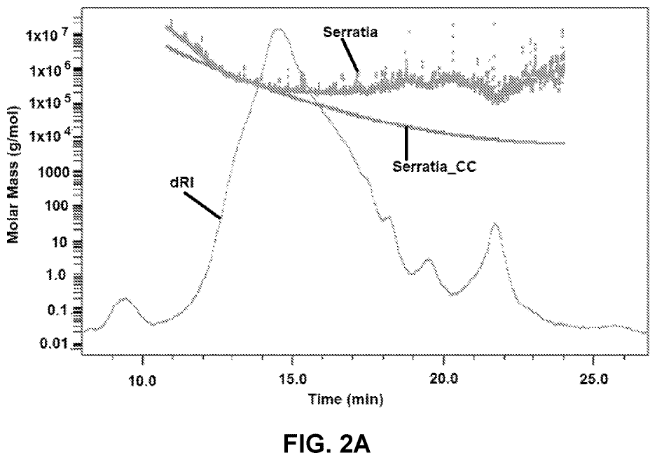

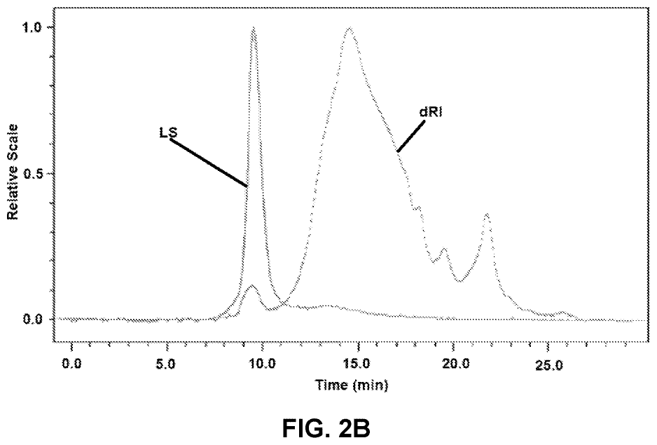

The methods used to prepare the Serratia polysaccharide for Hartwell and Shear's 1943 papers, where they showed (herein referred to as PS1) to be polysaccharide, peptide-free, endotoxin free (no LPS) comprised of hexoses, hexosamine and methyl pentose, with a lipid and a phosphate attached were also repeated herein. The Shear preparation involves large volumes of lime water, calcium hydroxide and calcium phosphate precipitations of the cell-free culture medium, where the precipitate is repeatedly redissolved in water and re-precipitated with 6 volumes of ethanol at pH 2, removing the salts from the polysaccharide. This laborious procedure resulted in a polysaccharide fraction referred to as PS1, which was chromatographed on a Sephacryl 400 SEC column, 30 cm×1 cm, using a 500 kDa dextran as a standard. (FIG. 1) PS1 gave a molecular weight of 250,000 as determined using dextran standards with refractive index and light scattering detection (FIGS. 2A-2B).

### Fractionation of PS1

Interestingly, in 1954, Rathgeb and Sylven attempted fractionation of Serratia polysaccharide obtained from MJ Shear. Their work repeated Shear's fractionation with addition of a 25% trichloroacetic acid soluble fraction precipitated with 50% ethanol, generating a tumor-active Fraction 1 containing hexose, hexosamine, methyl pentose, phosphate, and lipid. Using the soluble fraction from picric acid treatment on Fraction 1, no precipitate occurred with 50% Ethanol, and the supernatant was divided into 2 parts by 75% ethanol precipitation, 1B, a soluble fraction containing hexose, N-acetylglucosamine, methyl pentose, phosphate, and lipid, and 1A a precipitate, 91% glucan fraction. They concluded 2 polysaccharides were present, a glucan and an N-acetylglucosamine-containing fraction, neither of which alone had tumor necrotizing activity present in Fraction 1 and starting material.

### Methylation Linkage Analysis of PS1

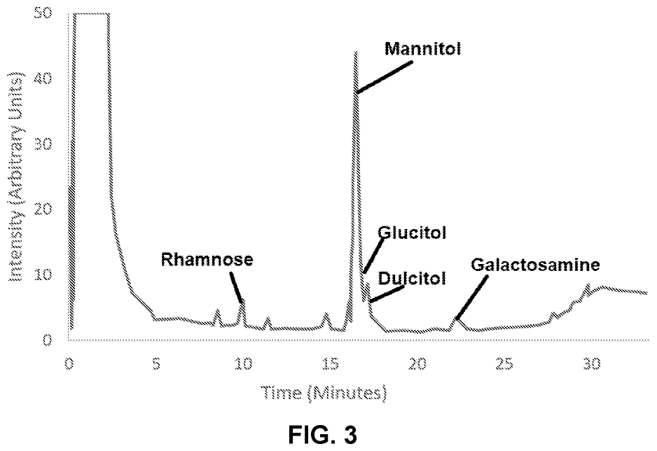

Anumula's method was used on PS1 and GC-MS spectra of terminal mannose, 2-linked mannose, 4-linked mannose, 2,6-branched mannose, and 4-linked N-acetylglucosamine were obtained, indicating the presence of a branched mannan (structural analysis in FIG. 3) and a GlcNAc containing compound.

### Mouse Tumor Assay Development

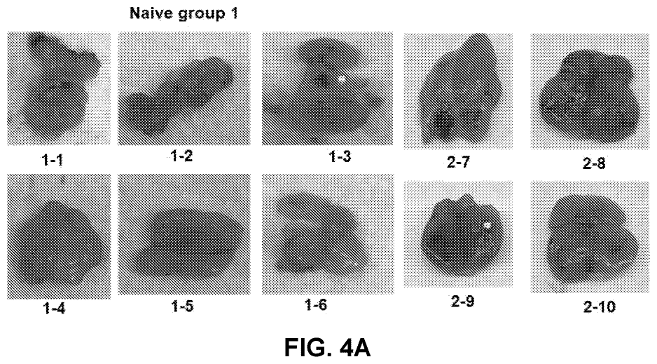

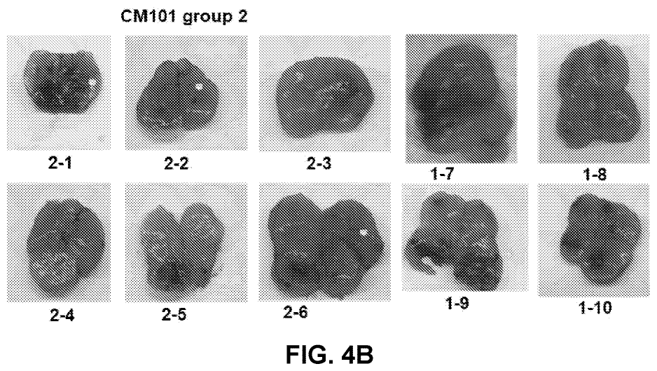

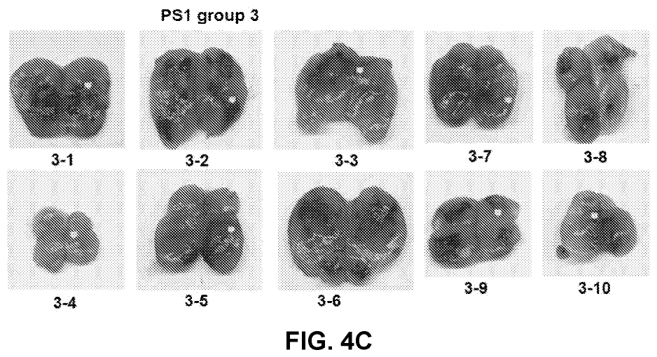

Murigenics, Inc. (Vallejo, CA) developed an assay for a transplantable CT26 mouse colon tumor model in Balb/C mice (FIGS. 4A-4C); and found potent tumor hemorrhage activity in CM101 and PS1 treated mice. FIG. 4A shows tumors from untreated (naïve) mice, no hemorrhage in the lobes of the tumors, some wound hemorrhage from excision. FIG. 4B shows tumors from mice treated with 1.2 μg/mouse of CM101, hemorrhage in 7 of 10 tumors. FIG. 4C shows tumors treated with PS1 Serratia polysaccharide, where so far one concentration, 4.8 μg/mouse, has been tested, where 9 of 10 tumors showed hemorrhage in all quadrants of the tumors and are continuing dose response studies of PS1. Thus, there is now a mouse tumor model for the tumor hemorrhage activity of PS1 based on the CT26 model using both male and female mice.

### Example 2: Determination of API Structure

Purification of PS1 from Serratia

Bacterial inocula are incubated to late log phase in Todd Hewitt Broth (THB) supplemented with 2 g/L of glucose and Na₂HPO₄. To avoid yeast mannans, yeast extract is dialyzed against culture media, the dialysis bag contents discarded and the dialysate is added to the medium, typically totaling 12 L. After 24 h fermentation at 37° C., the culture is autoclaved and cells removed by centrifugation. PS1 is present at a concentration of 1-15 mg/L in the supernatant fraction. An 0.45 μm transverse flow Pellicon filter removes remaining particles and cells. The filtrate is concentrated via a 50 kD or 100 kD cutoff Pellicon transverse flow filter from 12 L to 20 0 mL. The concentrate is extracted with 1-4 volumes of chloroform or dichloromethane (DCM). The lower chloroform or DCM layer is recovered for redistillation and conserved, while the emulsion layer between the water layer and chloroform/DCM layer is collected and was found to contain 90% of the activity. The water layer is discarded or frozen for further extraction. The emulsion layer is dried under an air stream at room temperature overnight. This can be done industrially with reduced pressure rotary evaporative scale up equipment. The residue is redissolved in water and precipitated with 5 volumes of ethanol, which precipitates polysaccharides, which are then redissolved in water following ethanol removal. This step is repeated three times, and the ethanol is recovered and conserved. The resulting PS1 polysaccharide is purified on Sephacryl 400 SEC chromatography as a 250 kDa broad peak as shown in FIG. 1.

### Polysaccharide Structure Determination

There are a set of established methods for studying polysaccharide structures, since they have limited amenability to direct mass spectrometry or NMR of the polymer. Carbohydrate analysis involves difficulties relating to isomers; for example, a hexasaccharide has potentially 1012 structures. In saccharide analysis, it remains necessary to use combined wet chemistry and mass spectrometric and NMR methodologies. Methylation analysis and GC-mass spectrometry are to discern linkages. For PS1, repeating sequence, branch spacing, anomerity, and the like, need to be established. Polysaccharides have a repeating oligosaccharide sequence. Since terminal mannose was detected, indicating a branched chain, the polymer can be treated with alpha and β-mannosidases to determine if sugar is released and perform sequential methylation analysis to begin to establish a branching pattern and sequence. Timed mannosidase experiments may need to be performed in case iterative enzymatic removal of mannose units peels the reducing end structure. Lectin affinity chromatography can be useful. Serratia has been cultured with ¹³C-glucose to obtain material for ¹³C and proton NMR structure analysis. The anomeric configuration of fragments can be discerned, in some cases, by proton NMR from the spin-spin couplings of the protons on carbon 1 and 2 of detected sugars which occur upfield. ß-anomer gives a value around 9 ppm, while alpha gives 2-3 ppm, although there is no published single method using NMR nor mass spectrometry that can parse the details of a 250,000 Da polysaccharide. Combinations of partial degradation, purification of oligosaccharide products, enzyme degradation, methylation linkage analysis, and MALDI-TOF and ESI-MS/MS and NMR on subunits isolated were used. Amino acids all have different masses except for leucine and isoleucine, simplifying polypeptide sequencing by mass spectrometry. DNA and RNA sequencing are routine. Polysaccharide sequencing is much more difficult. For example, glucose, mannose, and galactose all have the same mass. They occur in both linear and branched configurations, and their first carbon has a or ß anomeric configuration. They can be linked by the 2,3,4 or 6 positions when they occur in pyranose 6 membered rings, but furanose rings are also abundant in nature. Saccharide sequencing is not simple.

### Partial Degradation

For any polysaccharide containing amino sugars such as N-acetylgalactosamine, a repeat unit can be generated by hydrazinolysis-deamination. The reducing end after deamination elimination becomes a 2,5-anhydrosugar, which can be analyzed after reduction to 2,5-anhydrohexitol. Methods available for a mannan fraction are Smith degradation using periodic acid to cleave between vicinal hydroxyls in the polysaccharide. Since a 6 linked hexose was found, these moieties would be destroyed because of 2,3,4 free hydroxyls. The 2-linked sugars would cleave between 3 and 4 carbons, so this may be useful for generating oligosaccharides from the polymer. 4-linked galactosamine was also detected, which would be degraded between carbons 2 and 3. Partial acid hydrolysis is one avenue, followed by chromatography on P-2 SEC or on activated charcoal with ethanol gradients which can generate oligosaccharides useful for characterizing the polysaccharides. 6-linkages are more susceptible to acetolysis than other linkages, so this is a possible approach to partial degradation as in Sakakibara, et al. 1972 describing degradation of acetylated polysaccharides.

### Methylation Linkage Analysis

Anumula's 1992 methylation with colloidal sodium hydroxide and methyl-iodide in DMSO was used, followed by TFA hydrolysis, aldose reduction with NaBD4 to induce alditol asymmetry, acetylation of the free hydroxyls and GC/MS of the partially methylated alditol acetates (PMAA). U.S. Pat. No. 6,407,069 claims that the sugar composition of Streptococcus agalactica CM101 comprises a molar ratio of (1 mannose):(3 galactose):(1 glucose):(1 N-acetyl glucosamine):(1 N-acetyl galactosamine), a phosphodiester bond, and a hydrophobic group, molecular weight 270,000 kDa. PS1 from Serratia has a different sugar composition, which was examined by chemical characterization of each fraction. Although this Poisson distribution may show more than one polysaccharide in the peak, which can be separately tested for tumor hemorrhage, it is also likely that the active API is expressed across the peak. Interrogation of the sugar composition, sugar linkage analysis over the Sephacryl Peak, plus partial degradation to obtain oligosaccharides from the PS1 sample led to definition of a potential repeat structure, and, using the mouse tumor hemorrhage assay, assessment was conducted to determine whether each method of partial degradation leads to loss or retention of activity.

### PS1 Repeating Structure

Most biologically active polysaccharides, such as the anticoagulant heparin, have a repeating oligosaccharide structure where a rare sequence of a few sugars (a pentasaccharide in heparin) contains all of the biological activity. Polysaccharides are not homogeneous in molecular weight and have a Poisson range of sizes based on a repeat motif. A broad peak does not necessarily indicate a mixture. Note the Poisson peaks for Dextran 500 kDa and PS1 peaks in FIGS. 2A-2B. Until homogeneous biologically active fragments are obtained, performing direct mass spectrometry or NMR analysis on the intact saccharide is not useful. 2-D ¹³C/proton NMR using several pulse pattern techniques may show some evidence for the repeating structure. Therefore, fragmentation methods were used to obtain oligosaccharides amenable to mass spectrometry and NMR; later putting together the puzzle of the repeating structure, and activity enabling substitutions within the sequence. Preliminary data for PS1 PMAA showed terminal mannose, 2-linked, 4-linked, 2,6-branched mannose, and 4-linked N-acetylgalactosamine. FIG. 3 shows a sugar composition of PS1, by methanolysis and acetylation of the methyl glycosides followed by gas-liquid chromatography. Mannose is the main component, with a small amount of Rhamnose, galactose, glucose and galactosamine represented by their alditol acetates.

### Phosphodiester

Hartwell's work indicated that a phosphate and lipid were constituents of PS1. This is interesting since Streptococcus CM101 also had these components. Whether these entities are necessary for PS1 activity is not yet known. Phosphodiester bonds are relatively stable to alkali and acid, however phosphodiester linked mannose has been reported removed with pH 2.1 at 100° C. conditions. The Anumula methylation technique preserved phosphodiesters, thus it was possible to treat the methylation product with strong alkali, to which both glycosidic bonds and methyl ethers are stable, remove the phosphodiester, and remethylate with trideuteromethyl Iodide, which identified the phosphate location on an individual sugar moiety by mass spectrometric fragmentation.

### Hydrophobic Group

Fatty acids are usually bonded by esterification. The Anumula methylation does not remove ester groups, but Hakomori methylation cleaves esters. Therefore using the two methods and comparing results, substitution of a hydrophobic group on a particular sugar can be discerned. Also, using the Anumula methylation, subjecting half the sample to mild saponification, would reveal an ester substitution by a difference in the methylation pattern of one or more sugars. In the HCl-methanolysis procedure, the hydrophobic species can be extracted into a hexane fraction. The hexane fraction would contain methyl esters of hydrophobic acids, including fatty acids, hydroxy fatty acids, farnesyl, and other potential substituents, identified by GC-MS.

### Example 3: Binding Studies

### SP55/HP59 Receptor

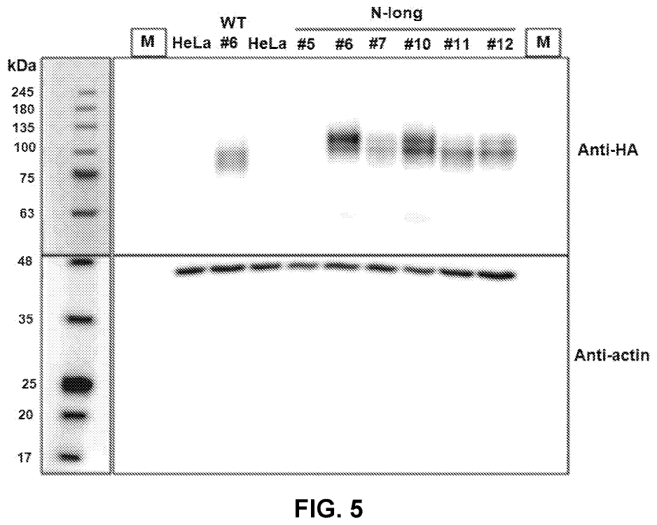

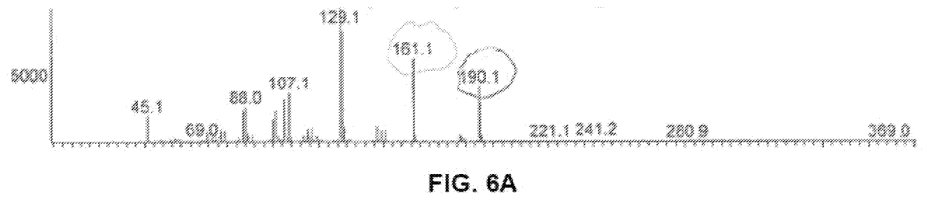

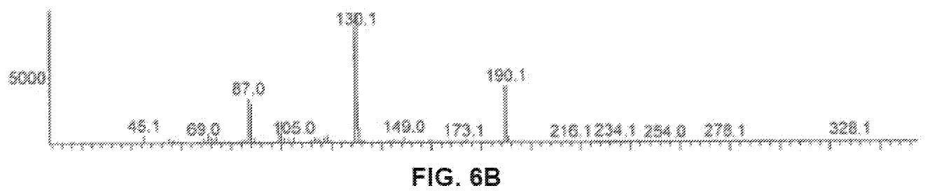

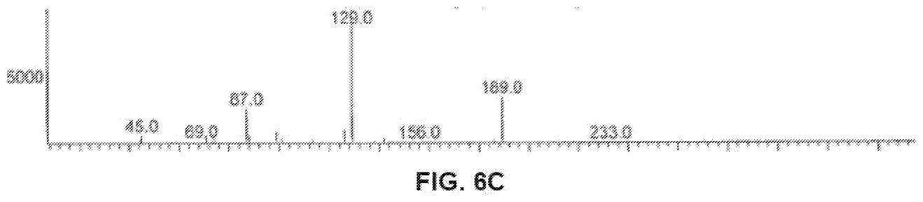

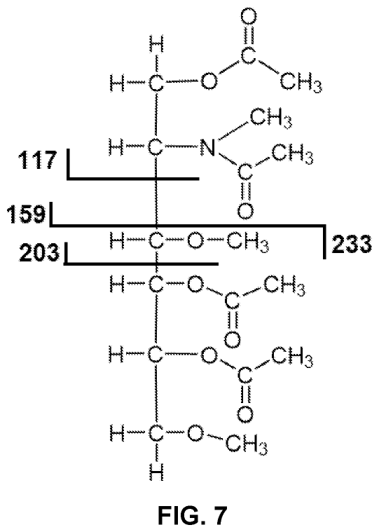

Cell based assays were performed including developing a stably transformed HeLa cell strain expressing SP55, the L22A, L23A mutant, on the cell surface (the dileucine motif directs the protein to lysosomal membranes, and the mutant protein traffics to the plasma membrane). A GFP-fusion of SP55 can be used for flow cytometric analysis, including the use of anti-GFP antibodies. Takematsu has since succeeded in cell expression of the HP59 human form and SP55 form with 98-106 HA tag in human U937 cells (FIG. 5). The HP59 form is highly expressed in clones 6 and 10, show as dimers in the gel with glycoform bands.

### Binding Studies of PS1 to SP55-HeLa and HP59-HeLa Cells

Biotinylated polysaccharides were used to bind to the SP55/HeLa cells, detected by fluorescent streptavidin in flow cytometry. Fragments of PS1 with reducing ends can be simply biotinylated for streptavidin ELISA or fluorescent probes to detect receptor specific cell binding by flow cytometry. A cell-based complement activation assay was established to assay the binding of PS1 and fragments to a receptor.

### Cell-Based (and Other) Alternative Pathway Activation Assays

Complement activation occurring on the surface of stably transformed cell lines expressing high-level SP55 or HP59 was investigated using immunohistochemical methods, specific monoclonal and polyclonal antibodies and/or labeled complement components to detect e.g., cell surface C3b, Factor Bb and C5b-9 neoantigen. Cell surface complement activation should result in release of C3a (des-Arg) and C5a which can be quantified using EIA and/or RIA. C3 depletion can be assayed using EsAC1,4-2 cells, i.e., sheep erythrocytes (Es), activated with antibody (A) and containing the assembled classical pathway C3-convertase enzyme, i.e., C1, C4 and C2. Assembly of C3 convertase enzyme on the surface of HP59 expressing cells was documented by incorporation of labeled Factor B and/or cleavage of Factor B to Bb in cell extracts run on SDS-PAGE. Should the use of stably transformed HP59 expressing cell lines prove problematic, agarose beads provide a receptive surface for alternative pathway complement activation and use of covalently linked (CNBr) HP59 provides one alternative. Other alternatives include cross-linking HP59 to complement cellular reagents, e.g., Es, where standard complement hemolytic assays can assess the effects on classical and alternative pathway activation. Because the major sugar is mannose, the possibility that the mannan binding protein MBL2 may be involved in the activation of complement was considered.

### Mouse Tumor Assay

Equal numbers of male and female Balb/c mice were procured for the studies. CT26 cells, 2×10⁶/100 μL, were injected subcutaneously in the left flank. Tumor measurements are done weekly until 300-500 μm size. For each test, groups of 3 female and 3 male mice were injected i.v. in the tail vein with 1.) vehicle control, normal saline, 5 L/g, 2.) Positive control clinical CM101, 50 μg/kg, 1.2 μg/ea. 3.) PS1 polysaccharide to 3 groups of 6 mice, 18 males and 18 females in 3 concentrations, 50 μg/kg, 100 μg/kg, and 200 μg/kg, 1.2-4.8 μg/mouse. Preliminary data was collected using one concentration 200 μg/kg for a single batch of PS1. A 250,000 Da protein is 250,000 g/mol, 250000 μg/μmol, 250 μg/nmol, 0.250 ng/pmol. 2.4 μg/mouse represents 10 μmol. The potency is extremely high. The amounts given are based on the weight of each mouse, usually around 25 g. After 4 hours, the mice are euthanized, the tumor is surgically removed and scored visually for vasculature leakage/hemorrhage. Images can be scanned for quantitation.

The resulting PS1 polysaccharide caused 90% tumor hemorrhage in a mouse model of colon tumor at a level of 77 nM. A transplantable CT26 mouse colon tumor model was used in Balb/c mice (FIGS. 4A-4C): revealing potent tumor hemorrhage activity in CM101 and PS1 treated mice. FIG. 4A shows tumors from untreated (naïve) mice, no hemorrhage in the lobes of the tumors, with minor wound hemorrhage from excision. FIG. 4B shows tumors from mice treated with 1.2 μg/mouse of CM101(7 nM), hemorrhage in 7 of 10 tumors. FIG. 4C shows tumors treated with PS1 Serratia polysaccharide, at 4.8 μg/mouse, (77 nM); 9 of 10 tumors showed hemorrhage in all quadrants.

### Example 4: Active Polysaccharide Preparation

The outcome of this research was a tumor hemorrhagic PS1 polysaccharide preparation that is specifically chemically defined, with an active saccharide sequence that elicits the well documented tumor hemorrhage phenomenon characterized already in 1947 by directly observed vascular disruption. The possibility remains that there is more than one polymer necessary for the activity, which would require 2 or more sugar sequences to activate complement upon cell binding, including the possibility of activity of the mannan binding protein MBL2. Receptors for the active polysaccharide(s) were identified.

Once identified, this preparation can be prepared at scale for use in pharmaceutical compositions. A S. marcescens strain can be obtained from a culture originally obtained from ATCC. This strain can be grown at either 37° C. or at room temperature, where it exhibits a red color on nutrient agar plates. Red colonies can be chosen from the room temperature S. marcescens for subculture and broth culture, allowing identification of desired bacteria and eliminating contamination from other organisms. Additional S. marcescens strains can be obtained from ATCC to determine whether a particular strain produces a higher level of PS1 polysaccharide in order to more efficiently scale production.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Al Jarousha, A. M., et al., An outbreak of Serratia marcescens septicaemia in neonatal intensive care unit in Gaza City, Palestine. J Hosp Infect, 2008. 70(2): p. 119-26.
2. Algire, G. H., et al, Vascular Reactions of Normal and Malignant Tissues Invivo.2. The Vascular Reaction of Normal and Neoplastic Tissues of Mice to a Bacterial Polysaccharide from Serratia Marcescens (Bacillus prodigiosus) Culture Filtrates. Journal of the National Cancer Institute, 1947. 8(2): p. 53-62.
3. Anumula, K. R. et al, A Comprehensive Procedure for Preparation of Partially Methylated Alditol Acetates from Glycoprotein Carbohydrates. Analytical Biochemistry, 1992. 203(1): p. 101-108.
4. Arumugham, R. G., et al., Structures of the asparagine-linked sugar chains of laminin. Biochim Biophys Acta, 1986. 883(1): p. 112-26.
5. Ashraf, J., et al., Enhancement of the Yu and Ledeen gas-liquid chromatographic method for sialic acid estimation: use of methane chemical ionization mass fragmentography. J Lipid Res, 1980. 21(8): p. 1137-41.
6. Barr, K., et al, Carbohydrate structures of three novel phosphoinositol-containing sphingolipids from the yeast Histoplasma capsulatum. Biochemistry, 1984. 23(23): p. 5589-96.
7. Beebe, S. P. et al, The treatment of experimental tumors with bacterial toxins. Journal of the American Medical Association, 1907. 49: p. 1493-1498.
8. Bruns, P., Die Heilwirkung des Erysipelas auf Geschwulste. Beitr. Klin. Chir., 1888. 3: p. 443-466.
9. Busch, W., Verhandlungen artzlicher Gesellschaften. 1868, Berl. Klin. Wochenschr.: Germany. p. 137-138.
10. Busch, W., Verhanlungen artzlicher Gesellshaften. 1866, Berl. Klin. Wochenschr.: Germany. p. 245-246.
11. Cann, Dr William Coley and tumour regression: a place in history or in the future. Postgrad Med J, 2003. 79: p. 672-680.
12. Chemsi, M., et al., An Outbreak of Serratia marcescens on the Neonatal Unit: Description and Investigations. J Infect Dis Ther, 2016. 4: p. 306.
13. Chien, J. L., et al., Characterization of gangliosides from bovine erythrocyte membranes. J Biol Chem, 1978. 253(11): p. 4031-5.
14. Cole, R. B., et al., 252Cf plasma-desorption mass spectrometry of lipid A from Enterobacter agglomerans. Rapid Commun Mass Spectrom, 1992. 6(10): p. 616-22.
15. Coley, W. B., A plea for more conservative treatment of sarcoma of the long bones. Journal of the American Medical Association, 1910. 54: p. 333-343.
16. Coley, W. B., I. Final Results in the X-Ray Treatment of Cancer, Including Sarcoma. Ann Surg, 1905. 42(2): p. 161-84.
17. Coley, W. B., II. Contribution to the Knowledge of Sarcoma. Ann Surg, 1891. 14(3): p. 199-220.
18. Coley, W. B., II. The Influence of Injury upon the Development of Sarcoma. Ann Surg, 1898. 27(3): p. 259-84.
19. Coley, W. B., IX. Carcinoma of the Breast, with a Round-Celled Sarcoma in the Submaxillary Region in the same Individual. Ann Surg, 1898. 27(1): p. 65-8.
20. Coley, W. B., Sarcoma of the Long Bones: The Diagnosis, Treatment and Prognosis, with a Report of Sixty-Nine Cases. Ann Surg, 1907. 45(3): p. 321-68.
21. Coley, W. B., The Treatment of Inoperable Sarcoma by Bacterial Toxins (the Mixed Toxins of the Streptococcus erysipelas and the Bacillus prodigiosus). Proc R Soc Med, 1910. 3 (Surg Sect): p. 1-48.
22. Coley, W. B., V. Sarcoma of the Clavicle: End-Results following Total Excision. Ann Surg, 1910. 52(6): p. 776-96.
23. Decker, W. K. and A. Safdar, Bioimmunoadjuvants for the treatment of neoplastic and infectious disease: Coley's legacy revisited. Cytokine Growth Factor Rev, 2009. 20(4): p. 271-81.
24. DeVore, R. F., et al., Phase I study of the antineovascularization drug CM101. Clinical Cancer Research, 1997. 3(3): p. 365-372.
25. Fehleisen, F., Uber die Zuchtung der Erysipelkokken auf kunstlichem Nahrboden und die Ubertragbarkeit auf den Menschen. 1882, Dtsch. Med. Wochenschr.: Germany. p. 553-554.
26. Fisher, S. J. et al, Carbohydrate structure of the major glycopeptide from human cold-insoluble globulin. J Supramol Struct, 1979. 11(3): p. 391-9.
27. Freeze, H. H., Lectin affinity chromatography. Curr Protoc Protein Sci, 2001. Chapter 9: p. Unit 9 1.
28. Fu, C. L., et al., Identification of a novel membrane protein, HP59, with therapeutic potential as a target of tumor angiogenesis. Clinical Cancer Research, 2001. 7(12): p. 4182-4194.
29. Hakomori, S., A Rapid Permethylation of Glycolipid, and Polysaccharide Catalyzed by Methylsulfinyl Carbanion in Dimethyl Sulfoxide. J Biochem, 1964. 55: p. 205-8.
30. Hartwell, J. L., et al, Chemical treatment of tumors. VII. Nature of the hemorrhage-producing fraction from Serratia marcescens (Bacillus prodigiosus) culture filtrate. Journal of the National Cancer Institute, 1943. 4: p. 107-122.
31. Hodges, L. C., et al, Structure of the oligosaccharide chains in human alpha 1-protease inhibitor. J Biol Chem, 1979. 254(17): p. 8208-12.
32. Hsieh, T. C., et al., Structure of a major glycophosphoceramide from tobacco leaves, PSL-I: 2-deoxy-2-acetamido-D-glucopyranosyl(alpha1 leads to 4)-D-glucuronopyranosyl(alpha1 leads to 2)myoinositol-1-O-phosphoceramide. Biochemistry, 1978. 17(17): p. 3575-81.
33. Jarnefelt, J., et al., Erythroglycan, a High Molecular-Weight Glycopeptide with Repeating Structure [Galactosyl-(1-]4)-2-Deoxy-2-Acetamido-Glucosyl(1-]3)]Comprising More Than One-Third of Protein-Bound Carbohydrate of Human Erythrocyte Stroma. Journal of Biological Chemistry, 1978. 253(22): p.8006-8009.
34. Kahler, H., et al, Chemical treatment of tumors. VII. Ultracentrifugal and electrophoretic analysis of the hemorrhage-producing fraction from Serratia marcescens (Bacillus prodigious) culture filtrate. Journal of the National Cancer Institute, 1943. 4: p. 123-129.
35. Karbach, J., et al., Phase I Clinical Trial of Mixed Bacterial Vaccine (Coley's Toxins) in Patients with NYESO- 1 Expressing Cancers: Immunological Effects and Clinical Activity. Clinical Cancer Research, 2012. 18(19): p. 5449-5459.
36. Laine, R. A. et al, Analysis of trimethylsilyl O-methyloximes of carbohydrates by combined gas-liquid chromatography-mass spectrometry. Anal Biochem, 1971. 43(2): p. 533-8.
37. Laine, R. A. et al, Chemistry of human erythrocyte polylactosamine glycopeptides (erythroglycans) as related to ABH blood group antigenic determinants. Adv Exp Med Biol, 1988. 228: p. 331-47.
38. Laine, R. A., A Calculation of All Possible Oligosaccharide Isomers Both Branched and Linear Yields 1.05×10(12) Structures for a Reducing Hexasaccharide—the Isomer-Barrier to Development of Single-Method Saccharide Sequencing or Synthesis Systems. Glycobiology, 1994. 4(6): p. 759-767.
39. Laine, R. A., Enhancement of detection for partially methylated alditol acetates by chemical ionization mass spectrometry. Anal Biochem, 1981. 116(2): p. 383-8.
40. Laine, R. A., et al., Monoglucosyloxyoctadecenoic acid—a glycolipid from Aspergillus niger. Biochemistry, 1972. 11(12): p. 2267-71.
41. Laine, R. A., et al., Non-reducing terminal linkage position determination in intact and permethylated synthetic oligosaccharides having a penultimate amino sugar: fast atom bombardment ionization, collisional-induced dissociation, and tandem mass spectrometry. Biol Mass Spectrom, 1991. 20(9): p. 505-14.
42. Maletzki, C., et al., Pancreatic cancer regression by intratumoural injection of live Streptococcus pyogenes in a syngeneic mouse model. Gut, 2008. 57(4): p. 483-91.
43. Matsuura, F., et al, Oligosaccharides accumulated in the kidney of a goat with beta-mannosidosis: mass spectrometry of intact permethylated derivatives. Arch Biochem Biophys, 1981. 211(1): p. 485-93.
44. Mccarthy, E. F., The toxins of William B. Coley and the treatment of bone and soft-tissue sarcomas. Iowa Orthop J, 2006. 26: p. 154-8.
45. Morillo, A., et al., A six-month Serratia marcescens outbreak in a Neonatal Intensive Care Unit. Enferm Infecc Microbiol Clin, 2016. 34(10): p. 645-651.
46. Nauts, H. C. et al, Coley toxins—the first century. Adv Exp Med Biol, 1990. 267: p. 483-500.
47. Nauts, H. C., et al, A review of the influence of bacterial infection and of bacterial products (Coley's toxins) on malignant tumors in man; a critical analysis of 30 inoperable cases treated by Coley's mixed toxins, in which diagnosis was confirmed by microscopic examination selected for special study. Acta Med Scand Suppl, 1953. 276: p. 1-103.
48. Nauts, H. C., et al, The treatment of malignant tumors by bacterial toxins as developed by the late William B. Coley, M.D., reviewed in the light of modern research. Cancer Res, 1946. 6: p. 205-16.
49. Nowotny, A., Antitumor Effects of Endotoxins, in Handbook of Endotoxin, Cellular Biology of Endotoxin. 1985. p. 389-448.
50. Ohta, M., et al., Trehalose-based oligosaccharides isolated from the cytoplasm of Mycobacterium smegmatis. Relation to trehalose-based oligosaccharides attached to lipid. Eur J Biochem, 2002. 269(13): p. 3142-9.
51. Puopolo, K. M., et al, Early-onset group B streptococcal disease in the era of maternal screening. Pediatrics, 2005. 115(5): p. 1240-6.
52. Rathgeb, P. et al, Chemical Studies on the Tumor-Necrotizing Agent from Serratia-Marcescens (Shears Polysaccharide). Jnci-Journal of the National Cancer Institute, 1954. 14(5): p. 1109-1117.
53. Rathgeb, P. et al, Fractionation Studies on the Tumornecrotizing Agent from Serratia-Marcescens (Shears Polysaccharide). Jnci-Journal of the National Cancer Institute, 1954. 14(5): p. 1099-1108.
54. Roger, G. H., Seances et Mem Soc de Biol Paris, 1892. 2: p. 573-580.
55. Rojas, J., et al., Pulmonary hemodynamic and ultrastructural changes associated with Group B streptococcal toxemia in adult sheep and newborn lambs. Pediatr Res, 1983. 17(12): p. 1002-8.
56. Rojas, J., et al., Studies on group B beta-hemolytic Streptococcus. II. Effects on pulmonary hemodynamics and vascular permeability in unanesthetized sheep. Pediatr Res, 1981. 15(6): p. 899-904.
57. Rosenberg, R. D. et al, Correlation between structure and function of heparin. Proc Natl Acad Sci USA, 1979. 76(3): p. 1218-22.
58. Rupley, J. A., The Hydrolysis of Chitin by Concentrated Hydrochloric Acid, and the Preparation of Low-Molecular-Weight Substrates for Lysozyme. Biochim Biophys Acta, 1964. 83: p. 245-55.
59. Sakakibara, K., et al., Acetolysis of dextran NRRL b1397. Preparation of trisaccharides containing (1 leads to 2)- and (1 leads to 6)-alpha-D-glucose linkages. Carbohydr Res, 1972. 25(2): p. 443-51.
60. Schwimmer, E., The Curative Effect of Erysipelas on Some Diseases (Translated from Hungarian). Gyogyaszat, 1887. 22: p. 353-355.
61. Shear, M. J., et al, Chemical treatment of tumors. VI. Method employed in determining the potency of hemorrhage producing bacterial preparations. Journal of the National Cancer Institute, 1943. 4: p. 99-105.
62. Shear, M. J., Chemical treatment of tumors. IX. Reactions of mice with primary subcutaneous tumors to injection of a hemorrhage-producing bacterial polysaccharide. Journal of the National Cancer Institute, 1943. 4: p. 461-476.
63. Shear, M. J., et al, Chemical treatment of tumors. V. Isolation of the hemorrhage producing fraction from Serratia marcesens (Bacillus prodigiosus) culture filtrate. Journal of the National Cancer Institute, 1943. 4: p. 81-97.
64. Soria, C., et al., [Serratia marcescens outbreak in Neonatal Intensive Care Unit: Guayaquil, Ecuador]. Rev Chilena Infectol, 2016. 33(6): p. 703-705.
65. Spronck, C. H. H., Tumeurs malignes et maladies infecticuses. Ann. Inst. Pasteur, 1892. 6: p. 683-707.
66. Sundell, H. W., et al., Isolation and identification of the group B streptococcal toxin CM101 from infants with sepsis. J Pediatr, 2000. 137(3): p. 338-44.
67. Thurman, G. B., et al., Acute inflammatory changes in subcutaneous microtumors in the ears of mice induced by Intravenous CM101 (GBS toxin). Journal of Cancer Research and Clinical Oncology, 1996. 122(9): p. 549-553.
68. Thurman, G. B., et al., Effects of group B Streptococcus toxin on long-term survival of mice bearing transplanted Madison lung tumors. J Cancer Res Clin Oncol, 1994. 120(8): p. 479-84.
69. Thurman, G. B., et al., Effects of Group-B Streptococcus Toxin on Long-Term Survival of Mice Bearing Transplanted Madison Lung-Tumors. Journal of Cancer Research and Clinical Oncology, 1994. 120(8): p. 479-484.
70. Thurman, G. B., et al., Induction of Lymphokine Production in Tumor-Bearing Mice by Gbs Toxin. Journal of Immunology, 1993. 150(8): p. A219-A219.
71. Townsend, R. R., et al., Carbohydrate structure of human fibrinogen. Use of 300-MHz 1H-NMR to characterize glycosidase-treated glycopeptides. J Biol Chem, 1982. 257(16): p. 9704-10.
72. Tsimberidou, A. M., et al., Phase I clinical trial of hepatic arterial infusion of paclitaxel in patients with advanced cancer and dominant liver involvement. Cancer Chemother Pharmacol, 2011. 68(1): p. 247-53.
73. Verheijen, F. W., et al., A new gene, encoding an anion transporter, is mutated in sialic acid storage diseases. Nat Genet, 1999. 23(4): p. 462-5.
74. Verneuil, De l'inoculation de l'erysipele comme moyen curatif. 1886, Union Med.: Paris. p. 217-221.
75. Voelz, A., et al., Outbreaks of Serratia marcescens in neonatal and pediatric intensive care units: Clinical aspects, risk factors and management. International Journal of Hygiene and Environmental Health, 2010. 213(2): p. 79-87.
76. Wiemann, B. et al, Coleys Toxins, Tumor-Necrosis-Factor and Cancer-Research—a Historical-Perspective. Pharmacology & Therapeutics, 1994. 64(3): p. 529-564.
77. Xue, J., et al., Determination of linkage position and anomeric configuration in Hex-Fuc disaccharides using electrospray ionization tandem mass spectrometry. Rapid Commun Mass Spectrom, 2004. 18(17): p. 1947-55.
78. Xue, J., et al, Enhancing MS (n) mass spectrometry strategy for carbohydrate analysis: A b2 ion spectral library. J Proteomics, 2015. 112: p. 224-49.
79. Yoon, E. et al, Linkage position determination in a novel set of permethylated neutral trisaccharides by collisional-induced dissociation and tandem mass spectrometry. Biol Mass Spectrom, 1992. 21(10): p. 479-85.
80. Zeligs, B. J., et al., Age-dependent susceptibility of neonatal rats to group B streptococcal type III infection: correlation of severity of infection and response of myeloid pools. Infect Immun, 1982. 37(1): p. 255-63.
81. Zhu, B. C. et al, Purification of acetyllactosamine-specific tomato lectin by erythroglycan-sepharose affinity chromatography. Prep Biochem, 1989. 19(4): p. 341-50.
82. Sayers, E. W. et al, Conformation of a Trimannoside Bound to Mannose-Binding Protein by NMR and Molecular Dynamics Simulations. Biophys. J., 2002, 82, p. 2683-2699.

1. A method for producing an anticancer polysaccharide, the method comprising:
(a) extracting Serratia polysaccharide PS1 from a culture at least one strain of Serratia marcescens;
(b) subjecting the PS1 to at least one chemical or enzymatic treatment to hydrolyze at least one moiety from the PS1.

2. The method of claim 1, further comprising chromatographically separating at least one fraction of the PS1 from the culture prior to performing step (b).

3. The method of claim 2, wherein chromatographically separating comprises size exclusion chromatography.

4. The method of claim 1, wherein the at least one chemical treatment comprises hydrazinolysis-deamination, Smith degradation using periodic acid, partial acid hydrolysis, acetolysis, or any combination thereof.

5. The method of claim 1, further comprising extracting the anticancer polysaccharide using a solvent.

6. The method of claim 5, wherein the solvent comprises chloroform, dichloromethane, or any combination thereof.

7. A compound produced by the method of claim 1.

8. The compound of claim 7, wherein the compound comprises a plurality of polysaccharide units selected from α1,6Manₚ-α1,4Manₚ-α1,4(α1,2Manₚ)Manₚ, α1,4Manₚ-α1,2-Manₚ-α1,4(α1,6Manₚ)Manₚ-α1,2-α1,6Manₚ, or any combination thereof.

9. The compound of claim 7, wherein the compound has a molar mass of about 250 kDa.

10. The compound of claim 7, wherein the compound further comprises at least one fatty acid covalently attached to at least one of the plurality of the polysaccharide units.

11. The compound of claim 10, wherein the at least one fatty acid is covalently attached via an ester linkage or a phosphodiester linkage.

12. The compound of claim 10, wherein the at least one fatty acid comprises decanoic acid, undecanoic acid, dodecanoic acid, tridecanoic acid, tetradecanoic acid, pentadecanoic acid, hexadecanoic acid, heptadecanoic acid, octadecanoic acid, hexadecenoic acid, octadecenoic acid, 3,OH-decanoic acid, 3,OH-5-dodecenoic acid, 3,OH-dodecanoic acid, methylenehexadecanoic acid, methyleneoctadecanoic acid, or any combination thereof.

13. A pharmaceutical composition comprising the compound of claim 7.

14. A method for treating a tumor in a subject, the method comprising administering to the subject the compound of claim 7.

15. The method of claim 14, wherein from about 2 mg to about 10 mg of the compound are administered to the subject per kg of body weight of the subject.

16. The method of claim 15, wherein the compound causes capillaries supplying the tumor to hemorrhage.

17. (canceled)

18. The method of claim 14, wherein the tumor comprises a bone tumor, a muscle sarcoma, a brain tumor, a breast tumor, a liver tumor, a gall bladder tumor, a colorectal tumor, a stomach tumor, a pancreatic tumor, an esophageal tumor, a bladder tumor, a prostate tumor, a uterine tumor, an ovarian tumor, an oral tumor, a salivary gland tumor, a kidney tumor, a skin tumor, a lung tumor, a prostate tumor, or any combination thereof.

19. The method of claim 16, wherein the tumor is a colorectal tumor and wherein at least 90% of the capillaries supplying tumor hemorrhage.

20. The method of claim 14, further comprising administering at least one additional treatment to the subject.

21. The method of claim 20, wherein the at least one additional treatment comprises surgery, radiation, administering at least one additional chemotherapeutic agent to the subject, or any combination thereof.

22.-28. (canceled)
